(19) United States
(12) Patent Application Publication
(10) Pub. No.: US 2010/0092970 A1
(43) Pub. Date: Apr. 15, 2010

(54) DETERMINING THE PHASE OF DUPLICATED CYP2D6 ALLELES

(75) Inventors: John L. Black, III, Rochester, MN (US); Dennis J. O'kane, Rochester, MN (US); David A. Mrazek, Rochester, MN (US); Whitney Erin Kramer, Rochester, MN (US); Denise L. Walker, Rochester, MN (US); Pamela K. Fisher, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 12/530,275

(22) PCT Filed: Mar. 7, 2008
(86) PCT No.: PCT/US2008/056281

§ 371 Date: Sep. 8, 2009

Related U.S. Application Data
(60) Provisional application No. 60/906,077, filed on Mar. 8, 2007.

(51) Int. Cl. C12Q 1/68

(52) U.S. Cl. 435/6

(57) ABSTRACT

This document relates to methods and materials involved in detecting duplicated CYP2D6 alleles (e.g., CYP2D6/CYP2D7−CYP2D6 arrangements) in mammals (e.g., humans).

### CROSS-REFERENCE TO RELATED APPLICATIONS

This application claims the benefit of U.S. Provisional Application Ser. No. 60/906,077, filed Mar. 8, 2007.

### BACKGROUND

1. Technical Field

This document relates to methods and materials involved in detecting and genotyping duplicated CYP2D6 alleles (e.g., CYP2D6 duplicated arrangements) in mammals (e.g., humans).

2. Background Information

Cytochrome P450 2D6 (CYP2D6) is a gene encoding an important Phase I drug metabolizing enzyme. It is highly variable and this variability impacts the function of the gene and its encoded enzyme. Included in this variability are the non-duplicated arrangement, the CYP2D6*5 arrangement where CYP2D6 is deleted, the CYP2D6 duplicated arrangement, the CYP2D6/CYP2D7−*5 arrangement, and the CYP2D6/CYP2D7−CYP2D6 duplicated arrangement where CYP2D6 is duplicated in tandem on one chromosome. A DNA sample may contain any two arrangements when the source DNA has two intact chromosomes.

### SUMMARY

This document provides methods and materials involved in detecting duplicated CYP2D6 alleles in mammals. The methods and materials described herein can be used to determine the CYP2D6 duplication genotype. The ability to determine CYP2D6 duplication genotypes can aid patient care because CYP2D6 allele function can regulate the activation or inactivation of many drugs in common use across medical specialties. When more than one CYP2D6 allele is present, knowing which allele is duplicated can allow the proper phenotype to be assigned. For example, an individual with two copies of *1 can be an extensive metabolizer while an individual with two copies of *4 can be a poor metabolizer.

This document is based, in part, on the discovery of allele specific primer extension (ASPE)-based assays and associated algorithms that can predict duplicated CYP2D6 alleles. This document also is based, in part, on the discovery of polymerase chain reaction (PCR)-based assays and ASPE-based assays that can be used to genotype individual CYP2D6 alleles that are in duplicated arrangement. Methods for detecting CYP2D6 genetic arrangements can allow genotypes, and associated phenotypes, to be accurately assigned, which can have a significant impact on patient care.

In general, one aspect of this document features a method for detecting a duplicated CYP2D6 allele in a mammal. The method comprises, or consists essentially of, (a) using oligonucleotide primers to amplify a nucleic acid of a CYP2D6 allele, and (b) comparing a ratio of signals obtained for non-wildtype and wildtype alleles to a control ratio for non-wildtype and wildtype alleles obtained using nucleic acid of non-duplicated, heterozygous CYP2D6 alleles to determine if the ratio is different from the control ratio, thereby indicating that the mammal contains a duplicated allele. The nucleic acid can be a nucleic acid of a CYP2D6*1 allele, a CYP2D6*2 allele, a CYP2D6*2A allele, a CYP2D6*3 allele, a CYP2D6*4 allele, a CYP2D6*6 allele, a CYP2D6*9 allele, or a CYP2D6*10 allele. The nucleic acid can be amplified using a multiplex based allele specific primer extension assay (e.g., a Luminex based allele specific primer extension assay).

In other aspect, this document features a method for detecting a duplicated CYP2D6 allele in a mammal. The method comprises, or consist essentially of, (a) amplifying a nucleic acid comprising a duplicated CYP2D6 allele, or a portion thereof, and (b) using the amplified nucleic acid to perform an allele specific primer extension assay. The allele specific primer extension assay can be a multiplex based allele specific primer extension assay (e.g., a Luminex based allele specific primer extension assay).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is an illustration of various CYP2D6 arrangements and approximate PCR primer locations including a non-duplicated arrangement (A), a CYP2D6 duplication arrangement (B), a CYP2D6*5 arrangement (C), a CYP2D6/CYP2D7−*5 arrangement (D), and a CYP2D6/CYP2D7−CYP2D6 duplication arrangement (E).

FIG. 2 is an illustration of a DNA sample with CYP2D6*1/*1 with duplication readout (A) and an illustration of a DNA sample with CYP2D6*1/*4 with duplication readout (B).

FIG. 3 contains genotyping results obtained using the Tag-It™ Mutation Detection Kit for Cytochrome P450-2D6 and associated software. The readout predicts a CYP2D6*1/*2 (not *2A) with duplication. Examination using the algorithm described in this disclosure predicts CYP2D6*1/CYP2D6*2+*2. The 2850T SNP is above +2 SD for the MFI for that SNP (Table 1), thus the allele defined by that SNP is duplicated.

FIG. 4 contains genotyping results obtained using the Tag-It™ Mutation Detection Kit for Cytochrome P450-2D6 and associated software. The readout predicts a CYP2D6*1/*9 with duplication. Examination using the algorithm described herein predicts CYP2D6*1+*1/CYP2D6*9. The MFI ratio for the 2613delAGA SNP is below −2 SD for that SNP (Table 1), thus the allele defined by that SNP is present in only one copy and the wild-type allele is duplicated.

FIG. 5 contains genotyping results obtained using the Tag-It™ Mutation Detection Kit for Cytochrome P450-2D6 and associated software. The readout predicts a CYP2D6*2A/*4 with duplication. Examination using the algorithm described herein (Example 1) predicts CYP2D6*2A+*2A/CYP2D6*4. The CYP2D6*2A SNPs are −1584G and 2850T. While the MFI ratio for 2850G is over +2 SD the MFI ratio for the −1584G SNP is not, although, it is near the upper limits of +2 SD for that SNP. Thus, CYP2D6*2A is predicted to be duplicated. The CYP2D6*4 SNPs are 100T and 1846A. While the MFI ratio 1846A SNP is below −2 SD, the MFI ratio for the 100T SNP is not, although, it approaches the lower limits of −2 SD for that SNP.

FIG. 6 contains genotyping results obtained using the Tag-It™ Mutation Detection Kit for Cytochrome P450-2D6 and associated software. The readout predicts a CYP2D6*1/*4 with duplication. Both SNPs defining the *4 allele (100T and 1846A) are well within the limits of ±2 SD. The methods described in the PCR phase of duplication approach (Example 2) revealed this sample to be a CYP2D6*1+*1/CYP2D6*4N+4. Consequently, the algorithm does accurately predict duplications on both chromosomes. This state can be difficult to differentiate from that seen in FIG. 7 where the algorithm fails to make a prediction. Thus, this type of result will require that the method used in PCR phase of duplication approach (Example 2) be initiated.

FIG. 7 contains genotyping results obtained using the Tag-It™ Mutation Detection Kit for Cytochrome P450-2D6 and associated software. The readout predicts a CYP2D6*1/*4 with duplication. Examination using the algorithm described herein does not accurately predict the duplicated allele. The MFI ratio for the 100T SNP is at the upper limit of +2 SD but the MFI ratio for the 1846A approaches the lower limit of −2 SD. These ambiguous results require that the method used in PCR-based phase of duplication approach (Example 2) be initiated.

FIG. 8 contains genotyping results obtained using the Tag-It™ Mutation Detection Kit for Cytochrome P450-2D6 and associated software. The PCR phase of duplication approach method was used to uniquely amplify the non-duplicated arrangement in genomic DNA. The genotype for the amplified allele is CYP2D6*4. No heterozygosity was seen because only one allele was amplified. Duplication and Deletion signals are seen because the primers for this amplicon also amplify the Rep 7 sequence which contains the annealing site for the duplication and deletion ASPE primers.

FIG. 9 is an illustration of two chromosomes with CYP2D6 duplications where different alleles are present on the two chromosomes (A and B). While other arrangements are possible, tandems repeats of identical or closely related alleles is the norm.

FIG. 10 contains illustrations of two chromosomes with duplications on each: a CYP2D6 duplication arrangement (A), and a CYP2D6/CYP2D7−CYP2D6 duplication arrangement (B).

FIG. 11 contains genotyping results obtained using the Tag-It™ Mutation Detection Kit for Cytochrome P450-2D6 and associated software. The PCR phase of duplication method (Example 2) was used to uniquely amplify the 3′ allele in the “typical” CYP2D6 duplicated arrangement in genomic DNA. The genotype for the amplified allele is CYP2D6*1. No heterozygosity was seen because only one allele was amplified. Duplication signal is seen because the primers for this amplicon also amplifies the Rep Dup sequence which contains the binding site for the duplication ASPE primer.

FIG. 12 is an illustration of one chromosome with a CYP2D6/CYP2D7−*5 arrangement (A) and the other with a typical CYP2D6 duplication arrangement (B).

FIG. 13 contains genotyping results obtained using the Tag-It™ Mutation Detection Kit for Cytochrome P450-2D6 and associated software. The PCR phase of duplication approach (Example 2) was used to uniquely amplify the 5′ allele in the CYP2D6 duplicated arrangement in genomic DNA. The genotype for the amplified allele is CYP2D6*1. No heterozygosity was seen because only one allele was amplified. Duplication signal is seen because the primers for this amplicon also amplifies the Rep Dup sequence which contains the binding site for the duplication ASPE primer.

FIG. 14 contains genotyping results obtained using the Tag-It™ Mutation Detection Kit for Cytochrome P450-2D6 and associated software. The PCR phase of duplication approach (Example 2) was used to amplify the 5′ allele in the CYP2D6 duplicated arrangement in genomic DNA. However, this genomic DNA sample also has a chromosome with a CYP2D6/CYP2D7−CYP2D6 arrangement. This results in amplification of the 5′ allele seen in that arrangement too. A duplication and deletion signal will be seen since these samples usually have a Rep 7 segment located after the 5′ 2D6/2D7 recombinant and the primers prime across that specific Rep 7 region. Also, heterozygosity (often for C100T and sometimes for G1846A, depending on the alleles present and the transition site from CYP2D6 to CYP2D7) will be seen in those SNPs amplified by the alpha amplicon of the Tag-It™ Mutation Detection Kit for Cytochrome P450-2D6. Allelic dropout of the SNPs located in the beta amplicon for the CYP2D6/CYP2D7 gene is observed since there is no annealing site for the Tm beta R primer in that allele.

### DETAILED DESCRIPTION

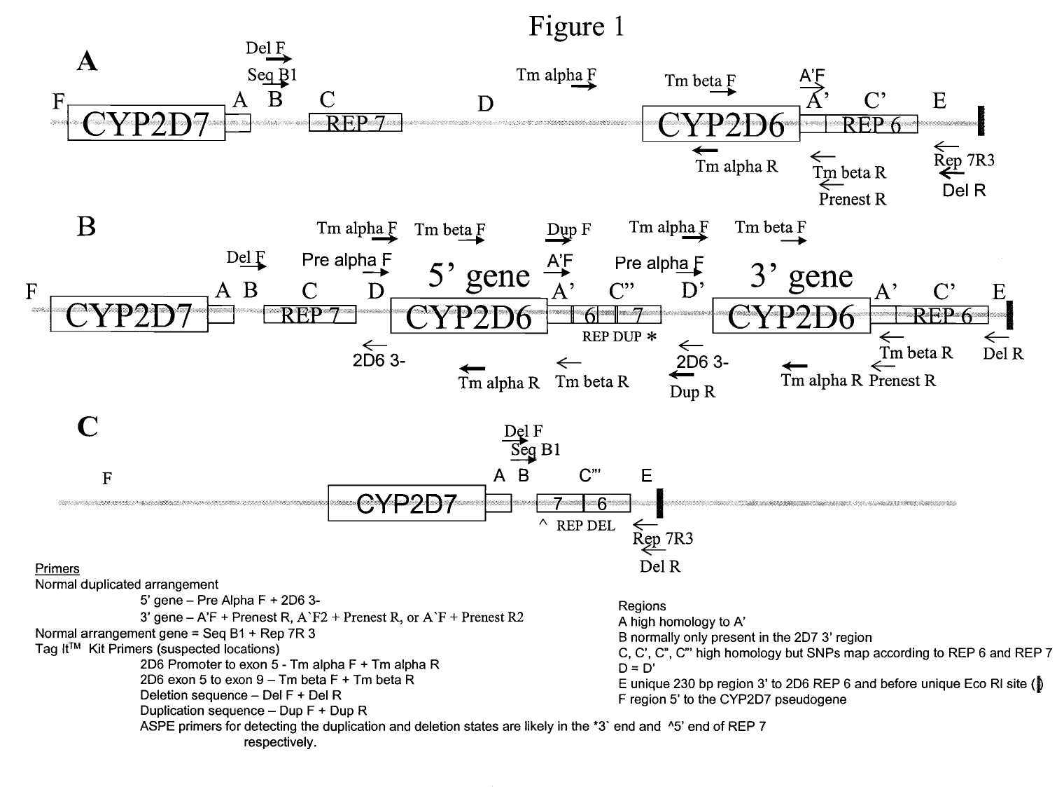

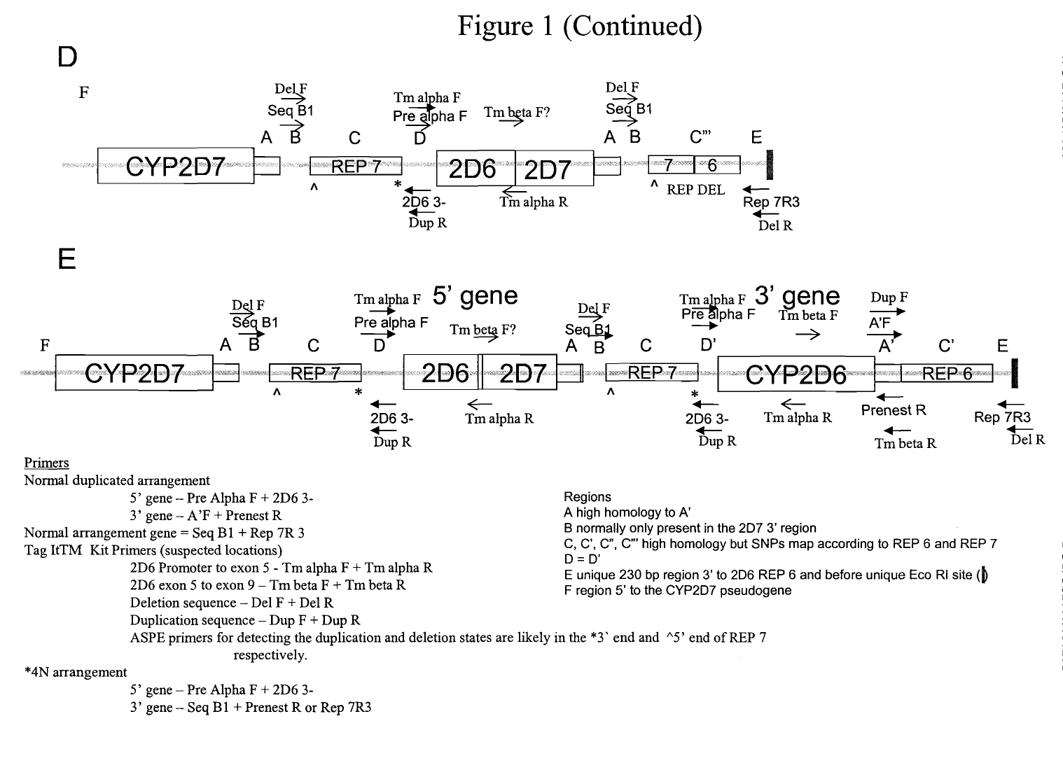

This document provides methods and materials related to detecting duplicated CYP2D6 alleles in mammals. A duplicated CYP2D6 allele can be any arrangement of a CYP2D6 gene that includes a duplication of a CYP2D6 allele or portion thereof. For example, a duplicated CYP2D6 allele can have a CYP2D6 duplication arrangement (FIG. 1B). In some cases, a duplicated CYP2D6 allele can have a CYP2D6/CYP2D7−CYP2D6 duplication arrangement (FIG. 1E). A CYP2D6 allele, or portion thereof, that is duplicated in a duplicated CYP2D6 allele can be any CYP2D6 allele. For example, a CYP2D6 allele that is duplicated can be a CYP2D6*1 allele or a CYP2D6*4 allele, or any portion thereof. The nomenclature of CYP2D6 alleles can be found on the world wide web at cypalleles.ki.se/cyp2d6.htm. A CYP2D6 nucleic acid sequence can be found in NCBI databases (ncbi.nlm.nih.gov; Accession Number M33388; SEQ ID NO:1).

Genomic DNA is typically used in an analysis of duplicated CYP2D6 alleles. Genomic DNA can be extracted from any biological sample containing nucleated cells, such as a peripheral blood sample or a tissue sample (e.g., mucosal scrapings of the lining of the mouth). Standard methods can be used to extract genomic DNA from a blood or tissue sample, including, for example, phenol extraction. Genomic DNA also can be extracted with kits such as the QIAamp® Tissue Kit (Qiagen, Valencia, Calif.) and the Wizard® Genomic DNA purification kit (Promega, Madison, Wis.).

A duplicated CYP2D6 allele can be detected by any appropriate DNA, RNA (e.g., Northern blotting or RT-PCR), or polypeptide (e.g., Western blotting or protein activity) based method. Non-limiting examples of DNA based methods include PCR methods (e.g., quantitative PCR methods and PCR methods described in Example 2), direct sequencing, fluorescence in situ hybridization (FISH), a Luminex based allele specific primer extension (ASPE) assay, such as that described in Example 1 or Example 2, and Southern blotting. In some cases, a duplicated CYP2D6 genotype can be determined using the Tag-It™ Mutation Detection Kit for P450-2D6 or the Roche Amplichip technology. In some cases, the phase of a duplicated CYP2D6 allele can be determined using an ASPE-based algorithm, such as that described in Example 1. In some cases, the phase of a duplicated CYP2D6 allele can be determined by isolating and genotyping a non-duplicated CYP2D6 allele and a 5′ and 3′ CYP2D6 duplicated allele. In some cases, a duplicated CYP2D6 allele can be detected based on altered CYP2D6 polypeptide function (e.g., decreased or no metabolism of one or more environmental chemicals or drugs). Any combination of such methods also can be used.

PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize complementary DNA (cDNA) strands.

Oligonucleotide primer pairs can be combined with genomic DNA from a mammal and subjected to standard PCR conditions, such as those described in Example 2, to amplify a CYP2D6 allele or portion thereof. For example, such a PCR reaction can be performed to amplify an entire duplicated CYP2D6 allele, or a portion of a duplicated CYP2D6 allele. The oligonucleotide primers having the nucleotide sequences set forth in SEQ ID NOs:2-8 are examples of primers that can be used to amplify nucleic acids containing duplicated CYP2D6 alleles, or portions thereof.

Amplified products can be separated based on size (e.g., in a slab-gel system or by capillary electrophoresis) and the appropriate detection system used to determine the size of the amplified product. In particular, an automated capillary electrophoresis system for separating and detecting the amplified products can be used, such as an Agilent bioanalyzer (Agilent Technologies, Santa Clara, Calif.). In some cases, detection of an amplification product of a particular size can indicate the presence and/or identity of a duplicated CYP2D6 allele.

A PCR amplification product containing a duplicated CYP2D6 allele, or portion thereof, can be analyzed further to genotype the allele. For example, such a PCR amplification product can be used in conjunction with the Tag-It™ Mutation Detection Kit for P450-2D6 (Tm Bioscience, Toronto, Ontario, Canada) to genotype the allele. In some cases, such an amplification product can be used in a Luminex based allele specific primer extension (ASPE) assay, such as that described in Example 2, to genotype a CYP2D6 allele (e.g., a duplicated CYP2D6 allele).

### Articles of Manufacture

Oligonucleotide primer pairs described herein can be combined with packaging materials and sold as articles of manufacture or kits for detecting duplicated CYP2D6 alleles. Oligonucleotide primers can be labeled with a detectable moiety, for example, a chemical tag allowing for colorimetric analysis, a fluorescent dye, or a radioisotope. In some cases, an article of manufacture can include sterile water, pharmaceutical carriers, buffers, antibodies, indicator molecules, DNA polymerase, nucleotides, and/or other useful reagents for detecting CYP2D6 alleles. Instructions describing how oligonucleotide primers can be used in an assay to detect duplicated CYP2D6 alleles can be included in such kits.

The invention will be further described in the following examples, which do not limit the scope of the invention described in the claims.

### EXAMPLES

### Example 1

### Determining the Phase of Duplicated CYP2D6 Alleles Using an Algorithm

The Luminex-based Tag-It™ Mutation Detection Kit for P450-2D6 (Tm Bioscience, Toronto, Ontario, Canada) was used to detect cytochrome P450-2D6 (CYP2D6) alleles. The CYP2D6 gene is highly variable, and examples of CYP2D6 gene arrangements include the non-duplicated arrangement, the CYP2D6*5 arrangement where CYP2D6 is deleted, the CYP2D6 duplicated arrangement, the CYP2D6/CYP2D7−*5 arrangement, and the CYP2D6/CYP2D7−CYP2D6 duplicated arrangement where CYP2D6 is duplicated in tandem on one chromosome (FIG. 1A-E). A DNA sample can contain any two arrangements assuming that two intact chromosomes are present.

Genotyping of genomic DNA was performed using the Tag-It™ Mutation Detection Kit for P450-2D6 and the associated software according to the manufacturer's instructions (Tm Bioscience). DNA also was prepared according to the manufacturer's instructions. The kit includes: PCR Primer Mix A and B (including dNTPs and the alpha and beta amplicon primers), ASPE Primer Mix (including dNTPs), Bead Mix (26 populations), 10×Wash Buffer, and Tag-It™ Data Analysis Software.

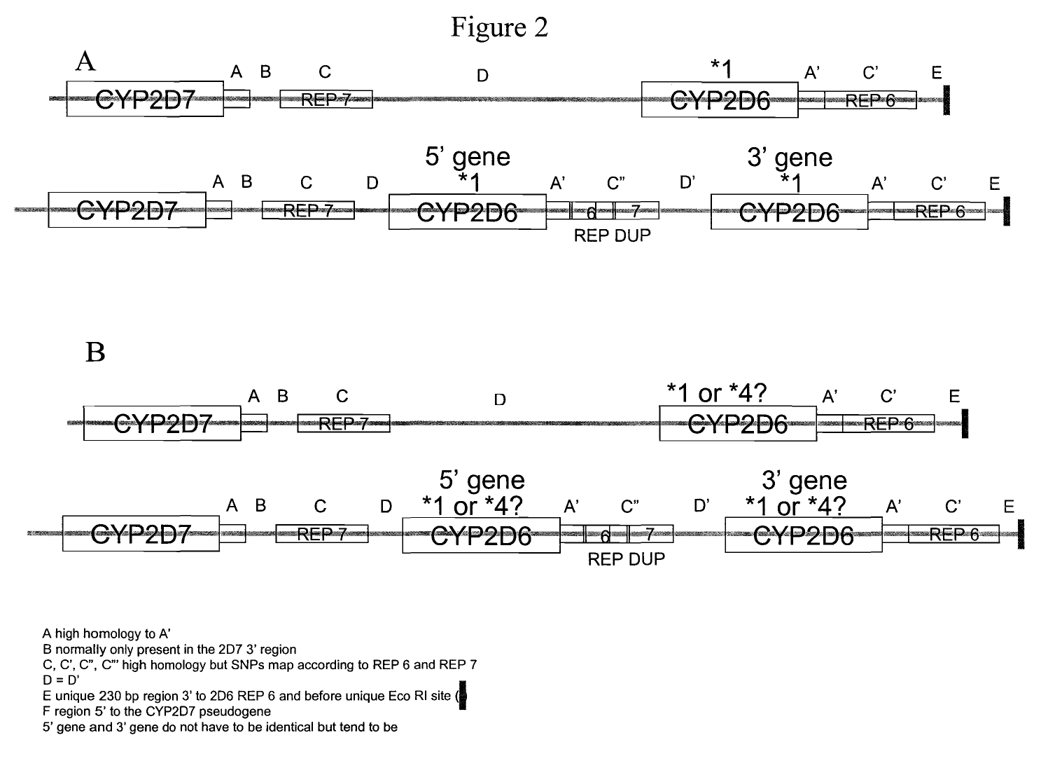

CYP2D6 duplications could be detected using the Luminex-based Tag-It™ Mutation Detection Kit for P450-2D6. When only one allele was detected in a sample with a duplication (e.g., *1/*1 with duplication), the phase of duplication could be determined because it was comprised of the only allele that was detected (FIG. 2A). When more than one allele was present, however, the kit did not determine which allele was duplicated, which prevented assignment of the proper phenotype (e.g., *1/*4 with duplication; FIG. 2B). In the example shown, either two copies of CYP2D6*1 or two copies of CYP2D6*4 could be present. This is clinically relevant when assigning phenotype because an individual with two copies of *1 is an extensive metabolizer while an individual with two copies of *4 is a poor metabolizer.

A method was developed for using data from the Tag-It™ Mutation Detection Kit for P450-2D6 to determine which allele was duplicated. The Tag-It™ Data Analysis Software was used to compare the mean fluorescent intensity (MFI) for both the wild-type and the mutant reaction in the analysis of individual SNPs. For heterozygous calls, the MFI ratio of mutant MFI/wild-type MFI fell between 0.3-0.7. It was hypothesized that a duplication of one allele would make the MFI for the SNP associated with that allele read higher than the SNP associated with the non-duplicated allele and visa versa, which would alter the MFI ratio in a predictable fashion. To test this hypothesis, the usual range of the MFI ratios was identified for several non-duplicated heterozygous results using SNPs that are part of commonly duplicated alleles. This was done by generating the mean and standard deviation for each MFI ratio in the heterozygous state at diagnostic SNP locations and calculating an MFI ratio range equal to the mean±2 standard deviations (±2 SD) using kits from lot RK004-0002 and lot RK0004-0003. The observed allelic ratio was calculated for SNPs located at −1584 (CYP2D6*2A), 100 (CYP2D6*4, *10), 1023 (CYP2D6*17), 1846 (CYP2D6*4), 2549 (CYP2D6*3), 1707 (CYP2D6*6), 2613 (CYP2D6*9), and 2850 (CYP2D6*2, *2A; Table 1). Samples with the duplicated arrangement were then studied using primers as described below in Example 2 to amplify the individual alleles present in the samples. Results of these experiments validated the hypothesis.

TABLE 1
Average MFI ratio ± 2 standard deviation ranges for heterozygous SNPs in
normal non-duplicated arrangement samples

| | *2A | *4 *10 | *17 | *4 | *3 | *6 | *9 | *2 *2A |
|---|---|---|---|---|---|---|---|---|
| | (−1584) | (100) | (1023) | (1846) | (2549) | (1707) | (2613) | (2850) |
| Mean | 0.56 | 0.45 | 0.54 | 0.51 | 0.51 | 0.46 | 0.54 | 0.51 |
| S.D. | 0.029851 | 0.029356 | 0.040591 | 0.019085 | 0.025238 | 0.028437575 | 0.012649 | 0.029877 |
| N | 44 | 53 | 7 | 42 | 35 | 47 | 40 | 69 |
| Range | 0.50-0.62 | 0.39-0.51 | 0.46-0.62 | 0.47-0.55 | 0.46-0.56 | 0.40-0.52 | 0.52-0.57 | 0.45-0.57 |

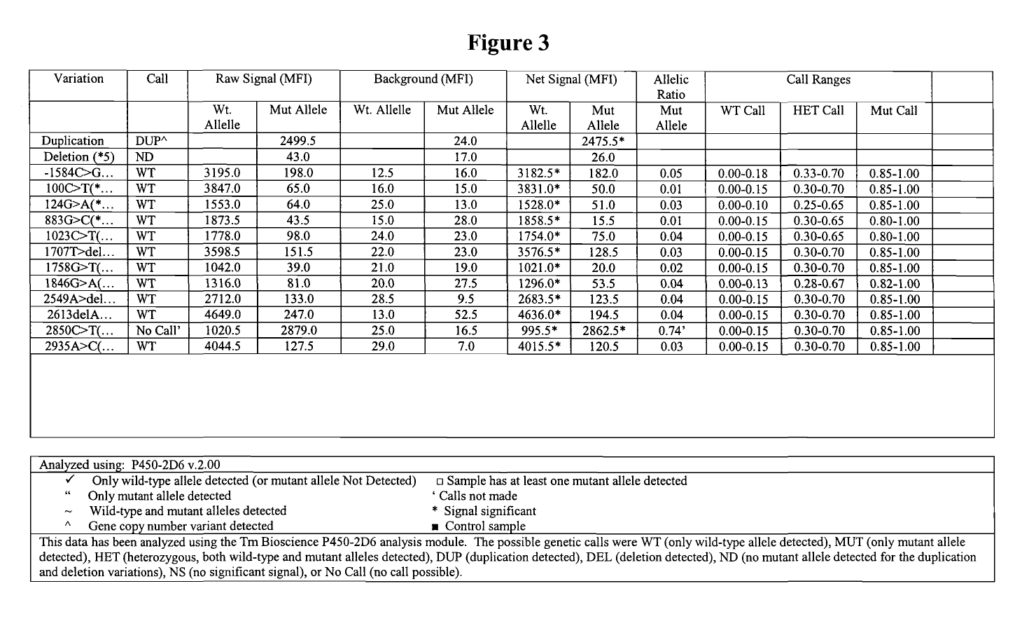

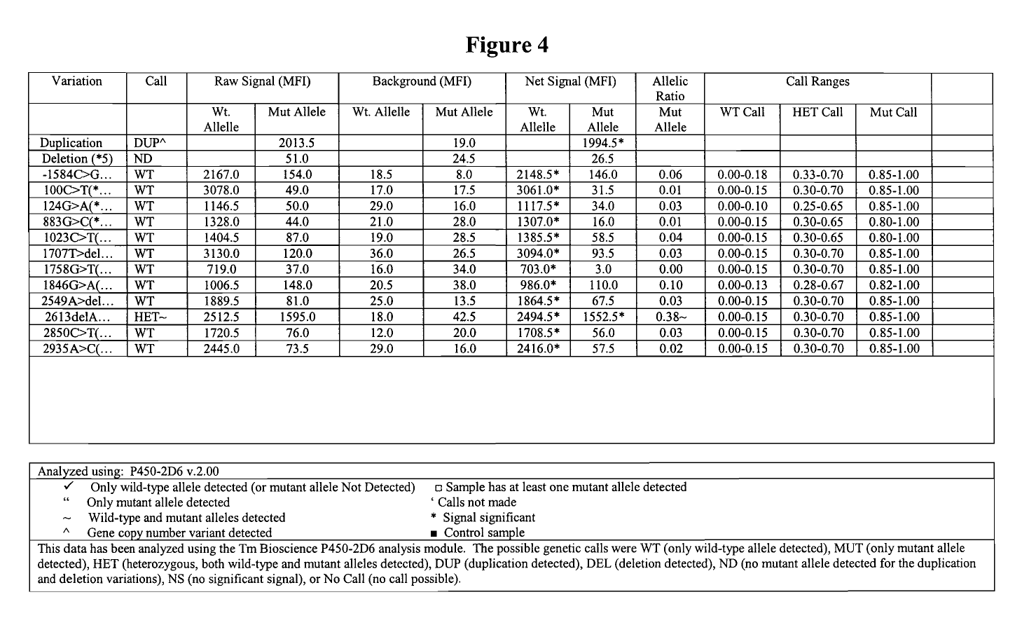

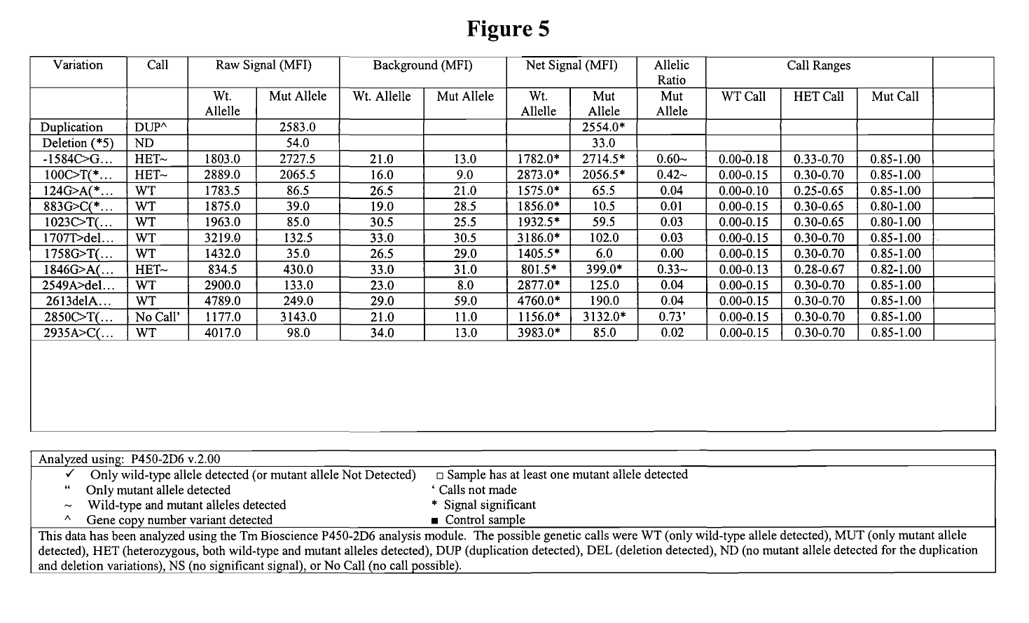

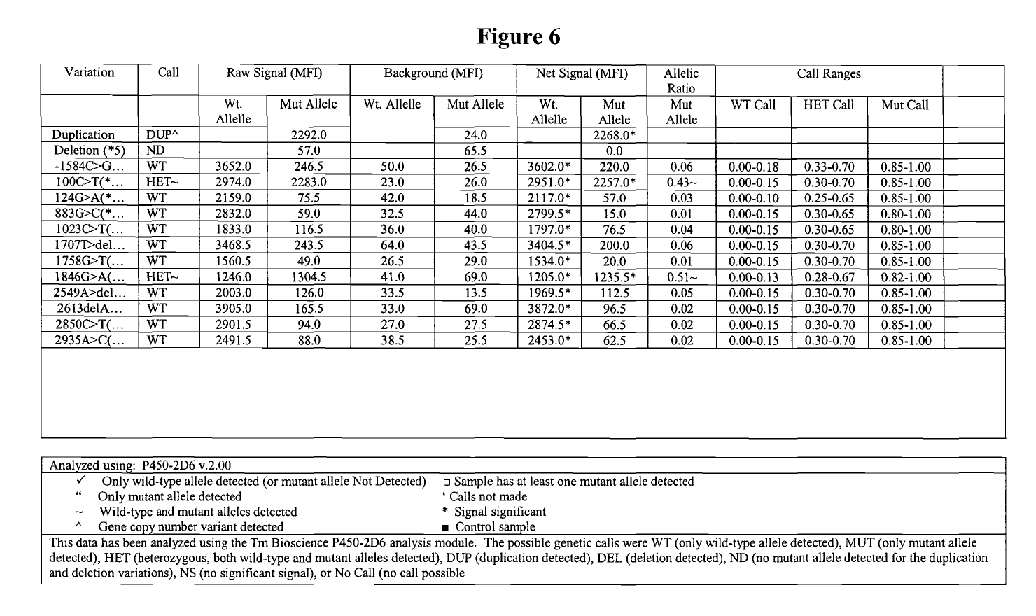

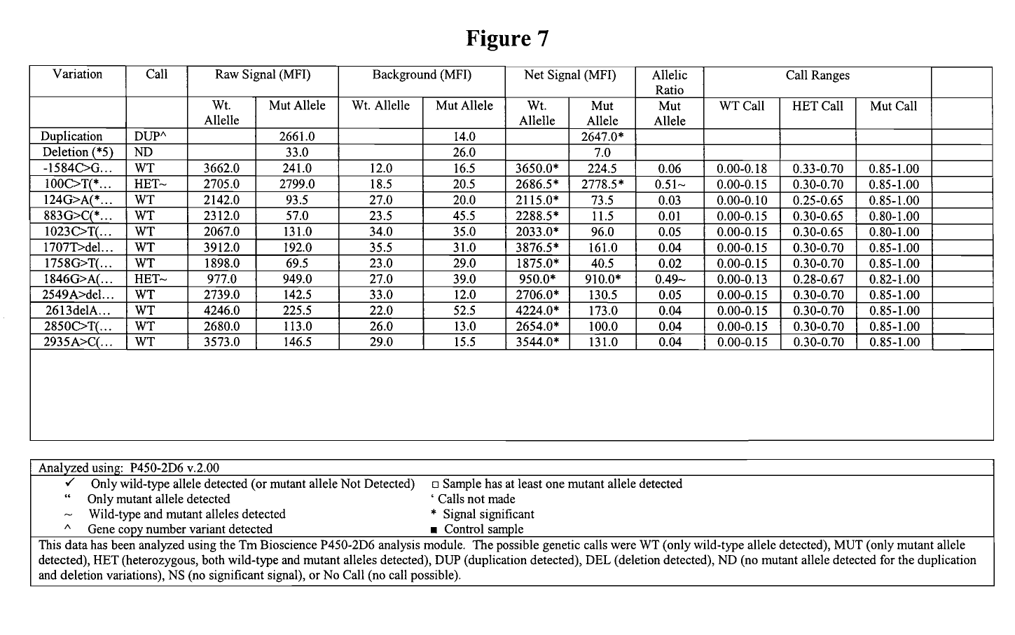

MFI intensities and ratios can vary from laboratory to laboratory. It may, therefore, be necessary for a given laboratory to test about 50 samples heterozygous for key SNPs to generate the laboratory's MFI ratio average±2 SD as shown in Table 1. In a sample where a duplication existed on one chromosome, the duplicated SNPs had an MFI ratio that was above the +2 SD shown in Table 1 (see also FIG. 3). When a duplicated nucleotide was on a *1 allele, the variant SNP had an MFI ratio that was below −2 SD (FIG. 4). In some cases, the SNPs determining a given duplicated or non-duplicated allele did not fall outside of ±2 SD. In these cases, where two or more SNPs defined an allele, the algorithm determined the duplicated or non-duplicated allele by first determining if any of the allele-defining SNPs were outside of ±2 SD, and then determining if the other SNPs defining an allele were near the limits of ±2 SD (FIG. 5). If so, and if both SNPs were in the same direction, e.g., both predicted a duplication, the duplicated allele was called on that basis. In cases where duplications of CYP2D6 existed on both chromosomes but the alleles were different (e.g., where one chromosome had *1 in duplicate and the other chromosome had *4 in duplicate), the Tag-It™ Mutation Detection Kit for P450-2D6 and associated software detected a duplication signal, but the MFI ratios did not fall outside of ±2 SD (FIG. 6). While MFI ratios solidly within the ±2 SD window suggested duplications on both chromosomes, these samples generally required further study using the PCR approach described in Example 2 to confirm the arrangement present. On occasion (about 10% of samples), the algorithm was not able to predict which allele was duplicated (FIG. 7). In these cases, individual PCR amplification (Example 2) of the non-duplicated allele, the 5′ allele duplicated allele and the 3′ duplicated allele was necessary, and these individual alleles were then identified by the Tag-It™ Mutation Detection Kit for P450-2D6.

### Example 2

### Determining the Phase of Duplicated CYP2D6 Alleles Using PCR

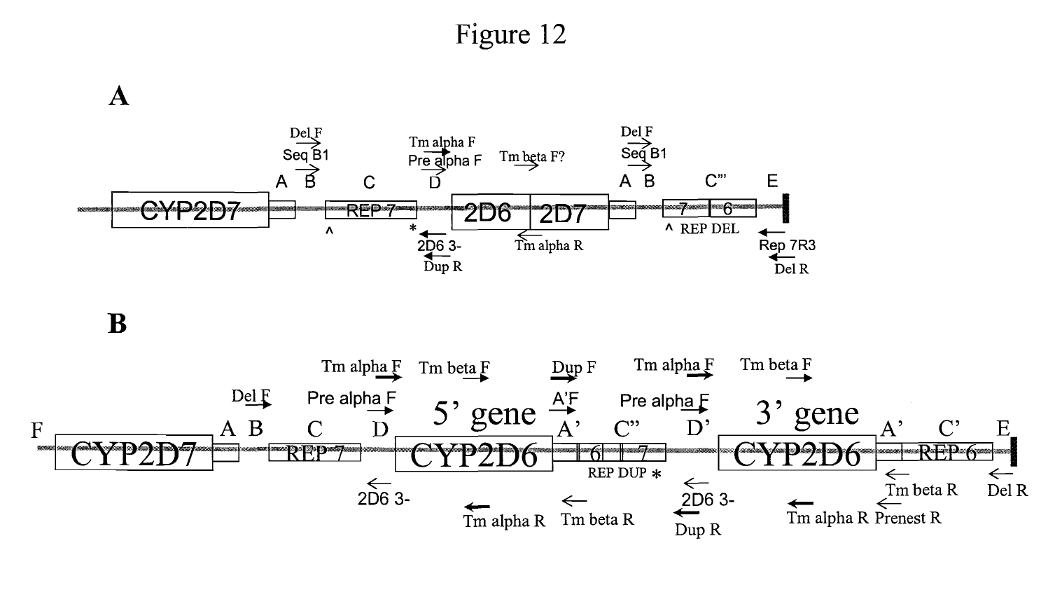

A PCR-based method was developed to determine the phase of duplicated CYP2D6 alleles. PCR reagents used to perform the assay included Takara LA Taq™ HS (5 U/μL). The storage buffer contained 20 mM Tris-HCL (pH 8.0), 100 mM KCl, 0.1 mM EDTA, 1 mM DTT, 0.5% Tween®20, 0.5% Nonidet P-40®, and 50% glycerol. The PCR buffer used was 10×LA PCR™ buffer (Mg²⁺ plus). The concentration of Mg²⁺ was 25 mM. An aqueous solution of dNTPs was used containing 2.5 mM of each dNTP and having a pH of about 7 to 9. In addition, betaine monohydrate was used (16.25 M, Mᵣ 135.16; Fluka Biochemika, Switzerland). The HPLC-purified primers (20 μg/μL) used to perform the amplifications are listed in Table 2. The approximate locations of the primers in CYP2D6 arrangements are illustrated in FIGS. 1, 9, 10, and 12.

TABLE 2
Primers used in the PCR-based phase of
duplication approach

| | | SEQ |
|---|---|---|
| Primer | Sequence | ID NO: |
| Seq B1 | GTCCCACACCAGGCACCTGTACT | 2 |
| Rep 7R3 | GAATTAGTGGTGGTGGGTGTTTG | 3 |
| Pre αF | TCACCCCCAGCGGACTTATCA | 4 |
| 2D6 3- | CCACAGCCCTCAATAAGTGAA | 5 |
| A′F | CACCACCCTCAGCCTCGTC | 6 |
| Prenest R | GACTGAGCCCTGGGAGGTAGGTA | 7 |
| Prenest R2 | TAGGTAGCCCTGGCCTATAGCTCCCTGACGCC | 8 |
| A′F2 | CCCTGGGAAGGCCCCATGGAAG | 9 |

Electrophoresis reagents used to perform the assay included agarose (catalog number 15510-027; Invitrogen, Carlsbad, Calif.), ethidium bromide (10 mg/mL; BioRad, Hercules, Calif.), and 1×TAE buffer, loading dye, and a 1 Kb Plus DNA ladder (Invitrogen). Exonuclease I (10 U/μL; USB Corporation, Cleveland, Ohio) and shrimp alkaline phosphatase (1 U/μL; USB Corporation) also were used.

An aqueous solution of betaine monohydrate was prepared (2.197 g/mL; 16.25 M). The solution was stored in a dark container at room temperature, and exposure to UV light was avoided. The primers, 10×buffer, and dNTP mixture were thawed. To isolate the 5′ 2D6 allele in the CYP2D6 duplicated arrangement (˜12052 by PCR product), a solution was prepared that contained 1.2 μL of 10×La PCR Buffer II (25 mM Mg²⁺), 2 μL of dNTP mixture (2.5 mM each), 6.48 μL of water, 1 μL of betaine (16.25 M), 0.5 μL of Pre alpha F primer, 0.5 μL of 2D6 3-primer, and 0.12 μL of TaKaRa LA Taq™ HS (5 U/μL) per 12 μL reaction volume. To isolate the 3′ 2D6 allele in the CYP2D6 duplicated arrangement (˜12103 by PCR product), a solution was prepared that contained 1.2 μL of 10×La PCR Buffer II (25 mM Mg²⁺), 2 μL of dNTP mixture (2.5 mM each), 6.48 μL of water, 1 μL of betaine (16.25 M), 0.5 μL of A′F primer or A′F2 primer, 0.5 μL of Prenest R or Prenest R2 primer, and 0.12 μL of TaKaRa LA Taq™ HS (5 U/μL) per 12 μL reaction volume. To isolate the non-duplicated arrangement 2D6 allele (˜15629 by PCR product) or the deleted CYP2D6*5 arrangement (˜3471 by PCR product), a solution was prepared containing 1.2 μL of 10×La PCR Buffer II (25 mM Mg²⁺), 2 μL of dNTP mixture (2.5 mM each), 6.48 μL of water, 1 μL of betaine (16.25 M), 0.5 μL of Seq B1 primer, 0.5 μL of Rep 7R3 primer, and 0.12 μL of TaKaRa LA Taq™ HS (5 U/μL) per 12 μL reaction volume. Each solution was mixed and centrifuged, and 11.8 μL of the master mix was added to each of an appropriate number of 0.2 mL thin-walled PCR tubes. DNA (0.2 μL) was added to each 0.2 mL tube containing master mix. DNA in EDTA reduces the robustness of the amplification. No more than 0.2 μL of DNA in EDTA was used for a 12 μL reaction. The PCR tubes were vortexed and centrifuged, and PCR amplifications were carried out. The following thermocycling conditions were used to amplify the 3′ (A′F/Prenest R2), 5′ (Pre alpha F/2D6 3-), nonduplicated, or deleted (Seq B1/Rep7R3) 2D6 allele in the CYP2D6 duplicated arrangement: 1 minute at 94° C.; 30 cycles of 10 seconds at 96° C., 30 seconds at 64° C., and 11 minutes at 68° C.; 10 minutes at 72° C.; and hold at 4° C. The following thermocycling conditions were used to amplify the 3′ 2D6 allele in the CYP2D6 duplicated arrangement (˜12138 by PCR product) when A′F2 and Prenest R were used as primer pairs: 1 minute at 94° C.; 30 cycles of 10 seconds at 96° C., 30 seconds at 62° C., and 11 minutes at 68° C.; 10 minutes at 72° C.; and hold at 4° C. The following thermocycling conditions were used to amplify the 3′ 2D6 allele in the CYP2D6 duplicated arrangement (˜12103 by PCR product) when A′F and Prenest R were used as primer pairs: 1 minute at 94° C.; 30 cycles of 10 seconds at 96° C., 30 seconds at 60° C., and 11 minutes at 68° C.; 10 minutes at 72° C.; and hold at 4° C. In some cases, the following thermocycling conditions were used to amplify the non-duplicated arrangement 2D6 allele (˜15629 by PCR product) or the deleted CYP2D6*5 arrangement (˜3471 by PCR product): 1 minute at 94° C.; 30 cycles of 10 seconds at 96° C., 30 seconds at 59° C., and 15 minutes at 68° C.; 10 minutes at 72° C.; and hold at 4° C.

PCR amplification was verified by agarose gel electrophoresis. A 0.5% agarose gel was prepared by melting 0.5 g of agarose in 100 mL of 1×TAE. The solution was allowed to cool for one minute before adding 10 μL of ethidium bromide. The agarose was poured into a large gel electrophoresis setup with a 20 well comb. The gel was allowed to solidify for 30 minutes. Three μL of each PCR reaction was mixed with three μL of loading dye and loaded into a well of the gel. The 1 Kb Plus DNA ladder (1.5 μL) also was mixed with three μL of loading dye and loaded into the gel. The gel was electrophoresed at 200 V for 45 minutes. Approximate sizes of PCR products corresponding to different CYP2D6 arrangements are listed in Table 3. Genomic DNA samples can contain any two of these arrangements. Table 3 can be used in interpreting electrophoresis results.

TABLE 3
Approximate sizes of PCR products amplified from samples containing
different CYP2D6 arrangements

| | | | Typical | | |
|---|---|---|---|---|---|
| Approximate | Normal Non- | Typical | CYP2D6 | CYP2D6/ | CYP2D6/CYP2D7− |
| PCR Product | duplicated | CYP2D6*5 | Duplication | CYP2D7+*5 | CYP2D6 |
| Size | Arrangement | Arrangement | Arrangement | Arrangement | Arrangement |
| Seq B1/Rep 7R3 | 15629 bp | 3471 bp | None | 3471 bp | 15629 bp (3′ |
| (designed to | | | | | allele is |
| amplify the | | | | | amplified) |
| normal non- | | | | | |
| duplicated | | | | | |
| arrangement) | | | | | |
| Pre alpha F/2D6 | None | None | 12052 bp | None | 13608 bp |
| 3 - (designed to | | | | | |
| amplify the 5′ | | | | | |
| allele of the | | | | | |
| typical duplicated | | | | | |
| arrangement) | | | | | |
| A′F/Prenest R | None | None | 12103 bp | None | None |
| (designed to | | | | | |
| amplify the 3′ | | | | | |
| allele of the | | | | | |
| typical duplicated | | | | | |
| arrangement) | | | | | |
| A′F2/Prenest R | None | None | 12138 bp | None | None |
| (designed to | | | | | |
| amplify the 3′ | | | | | |
| allele of the | | | | | |
| typical | | | | | |
| duplicated | | | | | |
| arrangement) | | | | | |
| A′F/Prenest R2 | None | None | 12086 bp | None | None |
| (designed to | | | | | |
| amplify the 3′ | | | | | |
| allele of the | | | | | |
| typical | | | | | |
| duplicated | | | | | |
| arrangement) | | | | | |

To a remaining 9 μL portion of each PCR reaction that was positive for an amplification product was added 0.5 μL (or 1.0 μL) of exonuclease I and 1.5 μL (or 4.0 μL) of shrimp alkaline phosphatase. In some cases, the resulting volume was about 14 μL. The 0.2 mL thin-walled tubes containing the solutions were placed into a thermocycler, and thermocycling was performed as follows to degrade unincorporated primers: 30 minutes at 37° C., 15 minutes at 99° C., and hold at 4° C. Individual amplicons were genotyped using the Tag-It™ Mutation Detection Kit for P450-2D6 (Tm Bioscience) according to the manufacturer's instructions, in either of two valid fashions. Either the max allotted amplicon volume was used when the ASPE was preformed directly on either the 5′, 3′, non-duplicated, or chimeric CYP2D6/2D7 amplicon (pretreated with exonuclease and shrimp alkaline phosphatase). Or, 5 μL of either the 5′, 3′, or non-duplicated amplicon (pretreated with exonuclease and shrimp alkaline phosphatase) was substituted for the genomic template in the TM alpha/beta amplification. The ASPE was subsequently preformed to genotype the isolated gene. The genotype of individual genes were then analyzed.

### CYP2D6 Non-Duplicated Arrangement Allele

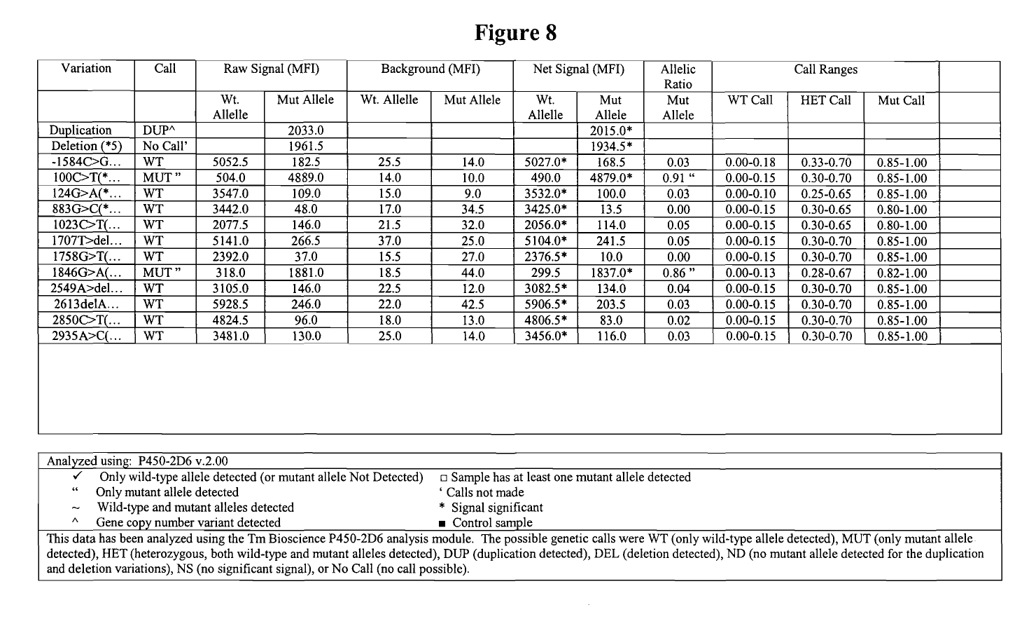

Due to the location of the primers for the non-duplicated arrangement allele (Seq B1 and Rep 7R3), the rep 7 region was amplified (FIG. 1A). Rep 7 has the priming site for the Duplication and Deletion ASPE primers of the Tag-It™ Mutation Detection Kit for P450-2D6. Thus, the readout was positive for both the duplication and deletion signals. The genotype for the sample was determined by the associated software or by eye. No heterozygosity was observed since only one allele was amplified and the sample was read as a homozygote for the allele present (FIG. 8).

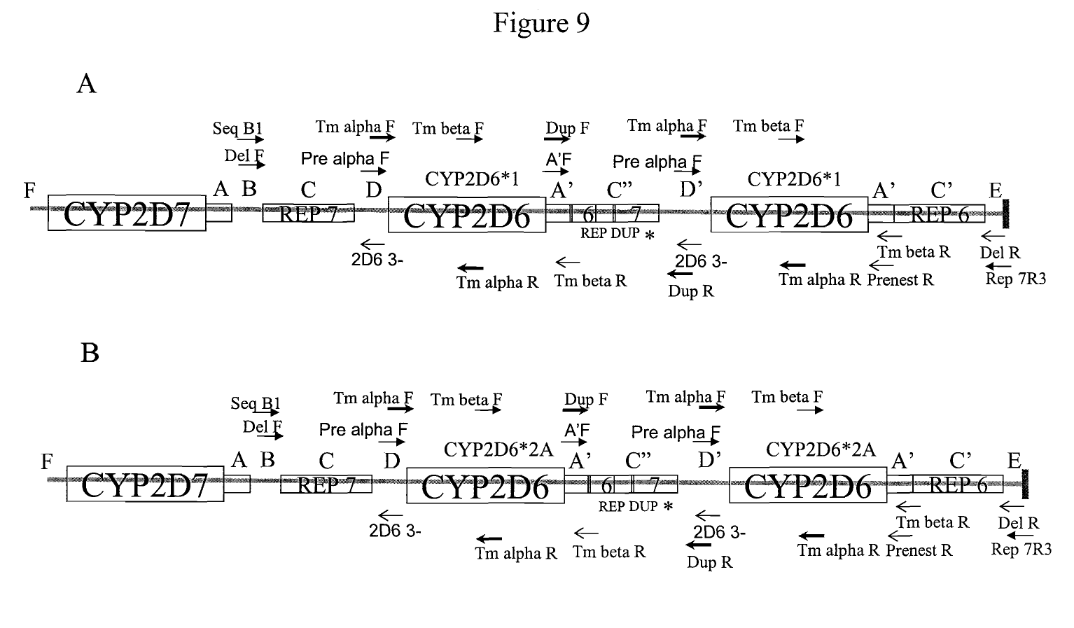

In situations where CYP2D6 duplication arrangements exist on both chromosomes, the PCR reaction may fail because the primers are potentially too far apart to yield a product with the cycling parameters described above (FIG. 9).

Where there was a CYP2D6/CYP2D7−CYP2D6 arrangement, the 3′ allele in this arrangement was amplified and the Tag-It™ Mutation Detection Kit for P450-2D6 yielded the genotype for that allele (FIG. 1E).

In cases where a CYP2D6/CYP2D7−*5 arrangement was present on one chromosome and a duplication arrangement was present on the other chromosome, ambiguous and weak readouts were common. This is because the Seq B1 primers prime two locations, and the shorter amplicon (which primes only the Rep Del region) dominates over a longer amplicon (FIG. 1D). If a product was produced at all, the Tag-It™ Mutation Detection Kit for P450-2D6 alpha amplicons were generated, but the reverse beta amplicons failed due to lack of the recognition site. Allelic dropout of beta amplicon SNPs is, therefore, expected.

In the unusual instance where a sample was tested which also had a CYP2D6*5 arrangement (FIG. 1C), a small amplicon across the Rep Del sequence was generated and the Tag-It™ Mutation Detection Kit for P450-2D6 gave only a deletion signal, if anything, and an otherwise unreliable genotype.

### CYP2D6 Duplicated Arrangement CYP2D6 Alleles

The 5′ and 3′ alleles were individually amplified and tested on the Tag-It™ Mutation Detection Kit for P450-2D6 product.

The 3′ Allele

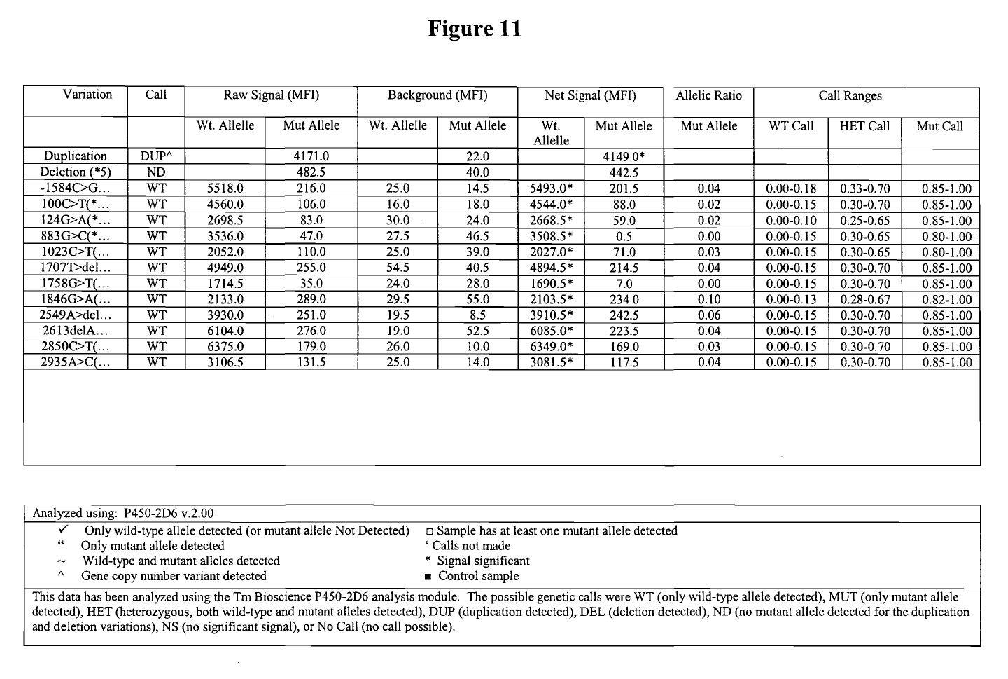

The primers (A′F and Prenest R) amplified the Rep Dup region of the sample and this yielded a duplication signal on the readout (FIG. 1B). The genotype for the sample was determined by the associated software or by eye. An additional feature of this primer set is that if the amplicon of the 3′ gene is used as the template for TM alpha beta amplification followed by the ASPE, it is very likely that the genotype will have a signal of both the duplication and deletion. This is due to the incomplete endonuclease digestion of the A′F primer. The endonuclease chips away the A′ specific 3′ end of the A′F primer leaving a fragment that is able to anneal to the A region. Thus, the remnant A′F primer anneals to A and generates a small fragment with Dup R during the alpha/beta amplification process. This amplifies the REP 7 region. Hence, this method will generate both the duplication and the deletion signal. The genotype/SNP output is unaffected. No heterozygosity was seen because only one allele was amplified and the sample was read as a homozygote for the allele present (FIG. 11).

In cases where a CYP2D6 duplication arrangement exists on both chromosomes (FIG. 9) and these duplications are identical in tandem, which is the usual situation, but the alleles on one chromosome are different from the alleles on the other chromosome, a heterozygotic state can be called by the Tag-It™ Mutation Detection Kit for P450-2D6 and associated software. This situation has not been observed.

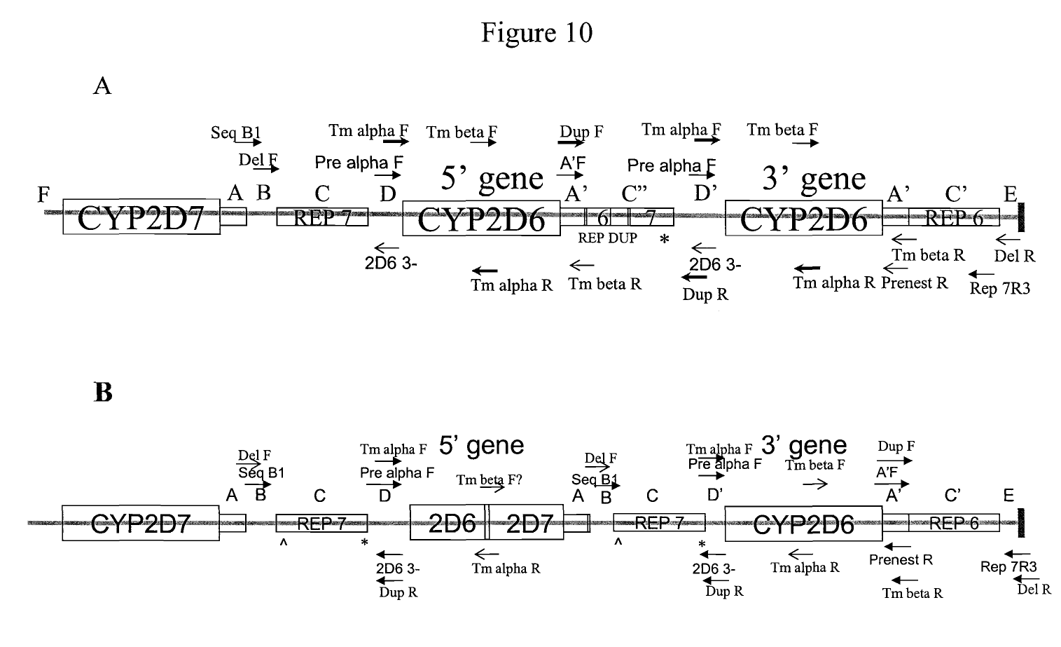

In instances where two CYP2D6/CYP2D7−CYP2D6 duplication arrangements exist on the two chromosomes in a genomic DNA sample, the DNA may not have an A′F primer annealing site and the PCR may fail to yield an appropriately sized amplicon. The Tag-It™ Mutation Detection Kit for P450-2D6 may not yield a valid genotype (FIG. 10B). This situation has not been observed.

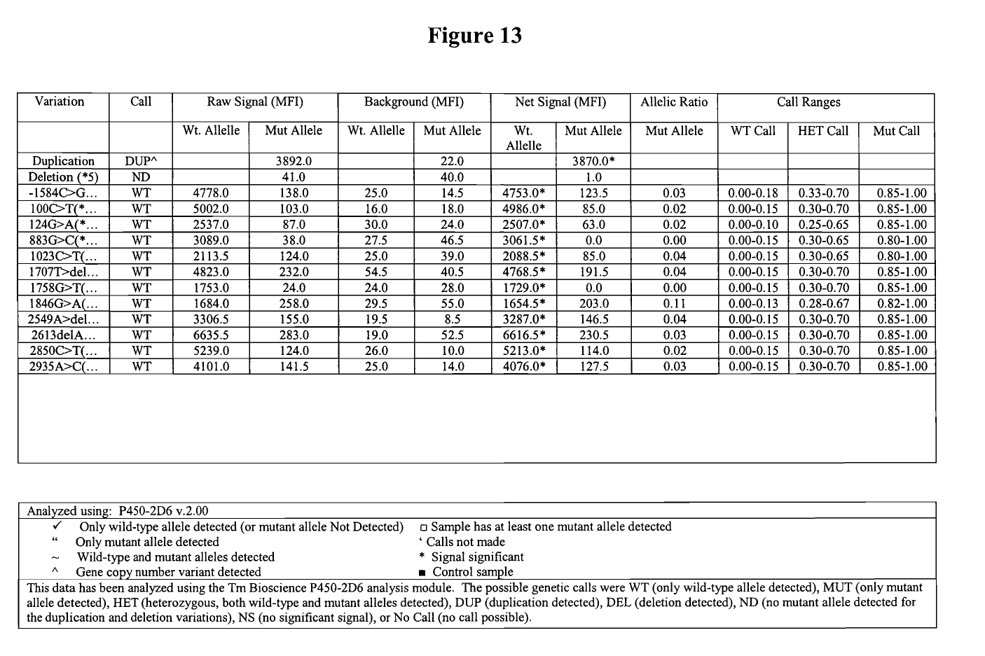

The 5′ Allele

Where a CYP2D6 non-duplicated arrangement and a CYP2D6 duplication arrangement situation existed, the primers (Pre alpha F and 2D6 3-) amplified the Rep Dup region of the sample and yielded a duplication signal on the readout. The genotype for the sample was determined by the associated software or by eye. No heterozygosity was seen in this instance because only one allele was amplified and the sample was read as a homozygote for the allele present (FIG. 13).

In instances where duplications exist on both chromosomes (FIG. 9) and these duplications are identical in tandem (which is the usual situation), but the alleles on one chromosome are different from the alleles on the other chromosome, a heterozygotic state can be seen. This situation has not been observed in the 50 samples tested.

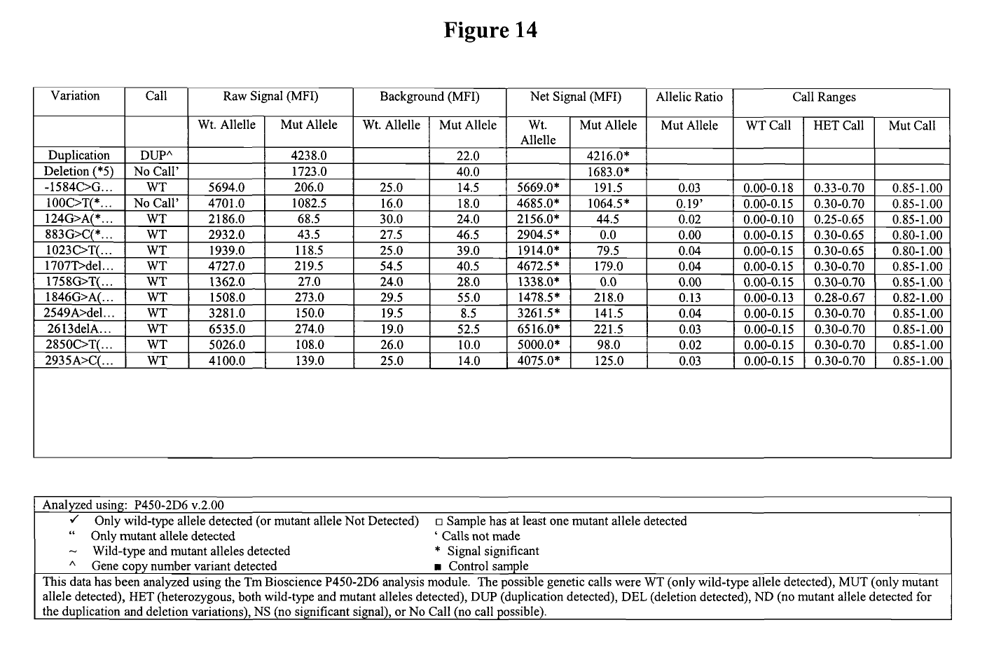

In situations where the duplication was a CYP2D6/CYP2D7−CYP2D6 arrangement and a CYP2D6 duplication arrangement (FIG. 10), a duplication and deletion signal were seen since these samples usually had a Rep 7 segment located after the 5′ 2D6/2D7 recombinant and the primers primed across that specific Rep 7 region. Also, heterozygosity (often for C100T and sometimes for G1846A, depending on the alleles present and the transition site from CYP2D6 to CYP2D7) was seen in those SNPs amplified by the alpha amplicon of the Tag-It™ Mutation Detection Kit for P450-2D6. Allelic dropout of the SNPs located in the beta amplicon for the CYP2D6/CYP2D7 gene was observed since there was no annealing site for the Tm beta R primer in that allele (FIG. 14).

For situations where both of the chromosomes had a CYP2D6/CYP2D7−CYP2D6 arrangement, a duplication and deletion signal may be seen since these samples usually have a Rep 7 segment located after the 5′ 2D6/2D7 recombinant and the primers prime across that specific Rep 7 region. Also, homozygosity or heterozygosity (often for C100T and sometimes for G1846A, depending upon the alleles present and upon the transition site from CYP2D6 to CYP2D7) can be seen in those SNPs amplified by the alpha amplicon of the Tag-It™ Mutation Detection Kit for P450-2D6. Allelic dropout of the SNPs located in the beta amplicon for the CYP2D6/CYP2D7 gene can be observed since there may be no annealing site for the Tm beta R primer in that allele (FIG. 10B).

### Example 3

### Reliability of the Algorithm

The majority of duplicated alleles could be predicted from the readout of genomic DNA using the algorithm (see Example 1).

SNPs such as −1584 in the case of a *2A duplication could not exceed +2 SD. However, the companion SNP of the *2A genotype, 2850, has proven a strong indicator of a *2A duplication. Often times, 2850 generated a “no call” because the allelic ratio exceeded the permissible value for a heterozygous read.

It was observed that cases where neither the duplicated nor the non-duplicated arrangement was CYP2D6*1 could be difficult to interpret using the algorithm. These appeared as many SNPs, which did or did not establish decipherable genotypes. In these cases, the PCR-based protocol could be used to ensure accurate genotyping.

Arrangements of >2 tandem CYP2D6 genes have been reported. These multiples may not be distinguishable from two tandem alleles since the 5′ and 3′ are typically identical in the CYP2D6 duplicated arrangement. However, if multiples of >2 tandem CYP2D6 are not identical alleles, the 5′ and 3′ are likely to have heterozygous signals, although this has not been observed.

Samples containing *4, suspected *4N +*4 or similar recombinants were observed to be particularly prone to giving ambiguous results.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting a duplicated CYP2D6 allele in a mammal, said method comprising (a) using oligonucleotide primers to amplify a nucleic acid of a CYP2D6 allele, and (b) comparing a ratio of signals obtained for non-wildtype and wildtype alleles to a control ratio for non-wildtype and wildtype alleles obtained using nucleic acid of non-duplicated, heterozygous CYP2D6 alleles to determine if said ratio is different from said control ratio, thereby indicating that said mammal contains a duplicated allele.

2. The method of claim 1, wherein said nucleic acid is a nucleic acid of a CYP2D6*1 allele, a CYP2D6*2 allele, a CYP2D6*2A allele, a CYP2D6*3 allele, a CYP2D6*4 allele, a CYP2D6*6 allele, a CYP2D6*9 allele, or a CYP2D6*10 allele.

3. The method of claim 1, wherein said nucleic acid is amplified using a multiplex based allele specific primer extension assay.

4. A method for detecting a duplicated CYP2D6 allele in a mammal, said method comprising (a) amplifying a nucleic acid comprising a duplicated CYP2D6 allele, or a portion thereof, and (b) using said amplified nucleic acid to perform an allele specific primer extension assay.

5. The method of claim 4, wherein said allele specific primer extension assay is a multiplex based allele specific primer extension assay.


SEQUENCE LISTING (9 sequences)

SEQ ID NO: 1 - DNA, 9432 bp, Homo sapiens
gaattcaagaccagcctggacaacttggaagaacccggtctctacaaaaaatacaaaattagctgggattgggtgcggtggctcatgcctataatcccagcactttgggagcctgaggtgggtggatcacctgaagtcaggagttcaagactagcctggccaacatggtgaaaccctatctctactgaaaatacaaaaagctagacgtggtggcacacacctgtaatcccagctacttaggaggctgaggcaggagaattgcttgaagcctagaggtgaaggttgtagtgagccgagattgcatcattgcacaatggaggggagccaccagcctgggcaacaagaggaaatctccgtctccaaaaaaaaaaaaaaaaaaaaaagaattaggctgggtggtgcctgtagtcccagctacttgggaggcagggggtccacttgatgtcgagactgcagtgagccatgatcctgccactgcactccggcctgggcaacagagtgagaccctgtctaaagaaaaaaaaaataaagcaacatatcctgaacaaaggatcctccataacgttcccaccagatttctaatcagaaacatggaggccagaaagcagtggaggaggacgaccctcaggcagcccgggaggatgttgtcacaggctggggcaagggccttccggctaccaactgggagctctgggaacagccctgttgcaaacaagaagccatagcccggccagagcccaggaatgtgggctgggctgggagcagcctctggacaggagtggtcccatccaggaaacctccggcatggctgggaagtggggtacttggtgccgggtctgtatgtgtgtgtgactggtgtgtgtgagagagaatgtgtgccctaagtgtcagtgtgagtctgtgtatgtgtgaatattgtctttgtgtgggtgattttctgcgtgtgtaatcgtgtccctgcaagtgtgaacaagtggacaagtgtctgggagtggacaagagatctgtgcaccatcaggtgtgtgcatagcgtctgtgcatgtcaagagtgcaaggtgaagtgaagggaccaggcccatgatgccactcatcatcaggagctctaaggccccaggtaagtgccagtgacagataagggtgctgaaggtcactctggagtgggcaggtgggggtagggaaagggcaaggccatgttctggaggaggggttgtgactacattagggtgtatgagcctagctgggaggtggatggccgggtccactgaaaccctggttatcccagaaggctttgcaggcttcaggagcttggagtggggagagggggtgacttctccgaccaggcccctccaccggcctaccctgggtaagggcctggagcaggaagcaggggcaagaacctctggagcagcccatacccgccctggcctgactctgccactggcagcacagtcaacacagcaggttcactcacagcagagggcaaaggccatcatcagctccctttataagggaagggtcacgcgctcggtgtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccaggtgagggaggaggtcctggagggcggcagaggtgctgaggctcccctaccagaagcaaacatggatggtgggtgaaaccacaggctggaccagaagccaggctgagaaggggaagcaggtttgggggacgtcctggagaagggcatttatacatggcatgaaggactggattttccaaaggccaaggaagagtagggcaagggcctggaggtggagctggacttggcagtgggcatgcaagcccattgggcaacatatgttatggagtacaaagtcccttctgctgacaccagaaggaaaggccttgggaatggaagatgagttagtcctgagtgccgtttaaatcacgaaatcgaggatgaagggggtgcagtgacccggttcaaaccttttgcactgtgggtcctcgggcctcactgcctcaccggcatggaccatcatctgggaatgggatgctaactggggcctctcggcaattttggtgactcttgcaaggtcatacctgggtgacgcatccaaactgagttcctccatcacagaaggtgtgacccccacccccgccccacgatcaggaggctgggtctcctccttccacctgctcactcctggtagccccgggggtcgtccaaggttcaaataggactaggacctgtagtctggggtgatcctggcttgacaagaggccctgaccctccctctgcagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggcaagcagcggtggggacagagacagatttccgtgggacccgggtgggtgatgaccgtagtccgagctgggcagagagggcgcggggtcgtggacatgaaacaggccagcgagtggggacagcgggccaagaaaccacctgcactagggaggtgtgagcatggggacgagggcggggcttgtgacgagtgggcggggccactgccgagacctggcaggagcccaatgggtgagcgtggcgcatttcccagctggaatccggtgtcgaagtgggggcggggaccgcacctgtgctgtaagctcagtgtgggtggcgcggggcccgcggggtcttccctgagtgcaaaggcggtcagggtgggcagagacgaggtggggcaaagcctgccccagccaagggagcaaggtggatgcacaaagagtgggccctgtgaccagctggacagagccagggactgcgggagaccagggggagcatagggttggagtgggtggtggatggtggggctaatgccttcatggccacgcgcacgtgcccgtcccacccccaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggtgggtgatgggcagaagggcacaaagcgggaactgggaaggcgggggacggggaaggcgaccccttacccgcatctcccacccccaggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgcggagcgagagaccgaggagtctctgcagggcgagctcccgagaggtgccggggctggactggggcctcggaagagcaggatttgcatagatgggtttgggaaaggacattccaggagaccccactgtaagaagggcctggaggaggaggggacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcatgtctgtccaggccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaaggtaggcctggcgccctcctcaccccagctcagcaccagcacctggtgatagccccagcatggctactgccaggtgggcccactctaggaaccctggccacctagtcctcaatgccaccacactgactgtccccacttgggtggggggtccagagtataggcagggctggcctgtccatccagagcccccgtctagtggggagacaaaccaggacctgccagaatgttggaggacccaacgcctgcagggagagggggcagtgtgggtgcctctgagaggtgtgactgcgccctgctgtggggtcggagagggtactgtggagcttctcgggcgcaggactagttgacagagtccagctgtgtgccaggcagtgtgtgtcccccgtgtgtttggtggcaggggtcccagcatcctagagtccagtccccactctcaccctgcatctcctgcccagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggtgcctgtggggagcccggctccctgtccccttccgtggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaactcgggtcccctgctcacgccctcgttgggatcatcctcctcagggcaaccccacccctgcctcattcctgcttaccccaccgcctggccgcatttgagacaggggtacgttgaggctgagcagatgtcagttacccttgcccataatcccatgtcccccactgacccaactctgactgcccagattggtgacaaggactacattgtcctggcatgtggggaaggggccagaatgggctgactagaggtgtcagtcagccctggatgtggtggagagggcaggactcagcctggaggcccatatttcaggcctaactcagcccaccccacatcagggacagcagtcctgccagcaccatcacaacagtcacctcccttcatatatgacaccccaaaacggaagacaaatcatggcgtcagggagctatatgccagggctacctacctcccagggctcagtcggcaggtgccagaacgttccctgggaaggccccatggaagcccaggactgagccaccaccctcagcctcgtcacctcaccacaggactggctacctctctgggccctcagggatgctgctgtacagacccctgaccagtgacgagttcgcactcagggccaggctggcgctggaggaggacacttgtttggctccaaccctaggtaccatcctcccagtagggatcaggcagggcccacaggcctgccctagggacaggagtcaaccttggacccataaggcactggggcgggcagagaaggaggaggtggcatgggcagctgagagccagagaccctgaccctagtccttgctctgccattaccccgtgtgaccccgggcccacccttccccacccttccccaccccgggcttctgtttccttctgccaacgagaaggctgcttcacctgccccgagtcctgtcttcctgctctgccttctggggctgtggcccttgctggcctggagccccaaccaagggcagggactgctgtcctccacgtctgtcctcaccgacataatgggctgggctgggcacacaggcagtgcccaagagtttctaatgagcatatgattacctgagtcctgggcagaccttcttagggaacagcctgggacagagaaccacagacactctgaggagccaccctgaggcctcttttgccagaggaccctacagcctccctggcagcagttccgccagcatttctgtaaatgccctcatgccagggtgcggcccggctgtcagcacgagagggacgttggtctgtcccctggcaccgagtcagtcagaagggtggccagggcccccttgggcccctccagagacaatccactgtggtcacacggctcggtggcaggaagtgctgttcctgcagctgtggggacagggagtgtggatgaagccaggctgggtttgtctgaagacggaggccccgaaaggtggcagcctggcctatagcagcagcaactcttggatttattggaaagattttcttcacggttctgagtcttgggggtgttagaggctcagaaccagtccagccagagctctgtcatgggcacgtagacccggtcccagggcctttgctctttgctgtcctcagaggcctctgcaaagtagaaacaggcagccttgtgagtcccctcctgggagcaaccaaccctccctctgagatgccccggggccaggtcagctgtggtgaaaggtagggatgcagccagctcagggagtggcccagagttcctgcccacccaaggaggctcccaggaaggtcaaggcacctgactcctgggctgcttccctcccctcccctccccaggtcaggaaggtgggaaagggctggggtgtctgtgaccctggcagtcactgagaagcagggtggaagcagccccctgcagcacgctgggtcagtggtcttaccagatggatacgcagcaacttccttttgaacctttttattttcctggcaggaagaagagggatccagcagtgagatcaggcaggttctgtgttgcacagacagggaaacaggctctgtccacacaaagtcggtggggccaggatgaggcccagtctgttcacacatggctgctgcctctcagctctgcacagacgtcctcgctcccctgggatggcagcttggcctgctggtcttggggttgagccagcctccagcactgcctccctgccctgctgcctcccactctgcagtgctccatggctgctcagttggacccacgctggagacgttcagtcgaagccccgggctgtccttacctcccagtctggggtacctgccacctcctgctcagcaggaatggggctaggtgcttcctcccctggggacttcacctgctctccctcctgggataagacggcagcctcctccttgggggcagcagcattcagtcctccaggtctcctgggggtcgtgacctgcaggaggaataagagggcagactgggcagaaaggccttcagagcacctcatcctcctgttctcacactggggtgtcacagtcctgggaagttcttccttttcagttgagctgtggtaaccttgtgagtttcctggagggggcctgccactacccttgggactccctgccgtgtgtctgggtctaactgagctctgaaaggagagagccccagccctgggccttccaggggaagccttacctcagaggttggcttcttcctactcttgactttgcgtctctgcagagggaggtgggaggggtgacacaaccctgacacccacactatgagtgatgagtagtcctgccccgactggcccatcctttccaggtgcagtcccccttactgtgtctgccaagggtgccagcacagccgccccactccaggggaagaggagtgccagcccttaccacctgagtgggcacagtgtagcatttattcattagcccccacactggcctgaccatctcccctgtgggctgcatgacaaggagagagaacaggctgaggtgagagctactgtcaacacctaaacctaaaaaatctataattgggctgggcagggtggctcacgcctgtaatcccagcactttgggaggccgagatgggtggatcacctgaggtcagatgttcgagaccagcctggccaacatggtgaaaccccgtctctactaaaaatacaaaaaattagctgggcgtggtggtgggtgcctgtaatcccagctactcaggaggctgaggcaggagaattgcttgaacctgggaggcagaggctgcagtgagccgagatcgcatcattgcactccagcctggtcaacaagagtgaaactgtcttaaaaaaaaaatctataattgatatctttagaaagataaaactttgcattcatgaaataagaataggagggtctaaaataaaaatgttcaaacacccaccaccactaattcttgacaaaaatatagtctgggtgccttagctcatgcctgtaatcccagcattttgggaggctaaggcaggaggattgtttgagcctaggaattc

SEQ ID NO: 2 - DNA, 23 bp, Homo sapiens
gtcccacaccaggcacctgtact

SEQ ID NO: 3 - DNA, 23 bp, Homo sapiens
gaattagtggtggtgggtgtttg

SEQ ID NO: 4 - DNA, 21 bp, Homo sapiens
tcacccccagcggacttatca

SEQ ID NO: 5 - DNA, 21 bp, Homo sapiens
ccacagccctcaataagtgaa

SEQ ID NO: 6 - DNA, 19 bp, Homo sapiens
caccaccctcagcctcgtc

SEQ ID NO: 7 - DNA, 23 bp, Homo sapiens
gactgagccctgggaggtaggta

SEQ ID NO: 8 - DNA, 32 bp, Homo sapiens
taggtagccctggcctatagctccctgacgcc

SEQ ID NO: 9 - DNA, 22 bp, Homo sapiens
ccctgggaaggccccatggaag